(19) United States
(12) Patent Application Publication
(10) Pub. No.: US 2026/0130940 A1
(43) Pub. Date: May 14, 2026

(54) POLYMER COMPOUND BINDING TO NATURAL KILLER CELLS AND SELECTIVELY KILLING CANCER CELLS INCLUDING SIALIC ACID AND METHOD FOR PRODUCING SAME

(71) Applicant: DONGGUK UNIVERSITY INDUSTRY-ACADEMIC COOPERATION FOUNDATION, Seoul (KR)

(72) Inventors: Kyobum Kim, Seoul (KR); Sungjun Kim, Seoul (KR); Ashok Kumar JANGID, Seoul (KR)

(73) Assignee: DONGGUK UNIVERSITY INDUSTRY-ACADEMIC COOPERATION FOUNDATION, Seoul (KR)

(21) Appl. No.: 19/443,480

(22) Filed: Jan. 8, 2026

Related U.S. Application Data
(63) Continuation of application No. PCT/KR2024/011488, filed on Aug. 5, 2024.

(51) Int. Cl. A61K 35/17; A61K 47/54; A61K 47/60; A61P 35/00

(52) U.S. Cl. CPC A61K 35/17; A61K 47/543; A61K 47/60; A61P 35/00

(57) ABSTRACT

Provided is a polymer compound. There may be provided a polymer compound including a hydrophobic moiety binding to a natural killer (NK) cell, a cancer cell recognition moiety, and a linker, in which the hydrophobic moiety is bound to one end of the linker and the cancer cell recognition moiety is bound to the other end of the linker, so as to recognize NK cells and cancer cells, in which the cancer cell recognition moiety selectively recognizes solid cancer cells including sialic acid.

### CROSS-REFERENCE TO RELATED APPLICATIONS

This is a continuation of International Patent Application PCT/KR2024/011488 (filed 5 Aug. 2024), the entire disclosure of which is hereby incorporated by reference herein.

### TECHNICAL FIELD

The present invention relates to a polymer compound which may be bound to a surface of a natural killer (NK) cell to enhance an anticancer function of the corresponding cell, in such a way that the polymer compound is synthesized, prepared, and injected to the body to selectively recognize and kill cancer cells including sialic acid, thereby dramatically improving a preventive or therapeutic effect on cancer.

### BACKGROUND ART

Immunotherapy is one of the most successful and innovative methods for treating various cancers. Natural killer (NK) cells are special effect cells having a unique ability to identify and remove tumor cells without disadvantages of T cells such as a lack of autologous T cells, an off-target toxicity, and a cytokine release syndrome, and have recently drawn attention as cells capable of replacing T cells.

However, there is a problem in that an ability to selectively target tumor cells is insufficient due to an immunosuppressive tumor microenvironment (TME) and a lack of specific cancer-targeting ligands of NK cells, thus resulting in a decrease in an efficacy of immunotherapy.

Chimeric antigen receptor (CAR)-based genetic engineering is one of the technologies which may overcome a target limitation of NK cells and improve a therapeutic performance, and clinical trials are underway around the world. As interest and demand for CAR-NK cells increase, problems related to CAR-based engineering are also emerging.

Specifically, a process of preparing genetically engineered CAR-NK cells suffers from low infection efficiency, and a unique biological activity of NK cells for tumors may be reduced due to unpredictable mutagenesis. Accordingly, there is a demand for the development of new technologies which do not depend on genetic modification.

As an alternative to overcome limitations associated with a production of CAR-NK cells, surface engineering technology of NK cells is being studied. As a related technique, a technique of targeting CD22 by modifying a surface of NK cells through glycoengineering has been developed, but an expression of a target ligand through glycoengineering is entirely dependent on intracellular metabolism, thus causing a problem in that precise control becomes difficult.

### Technical Problem

One technical object of the present invention is to provide a polymer compound capable of binding to NK cells.

Another technical object of the present invention is to provide a polymer compound capable of recognizing a target cancer cell.

Still another technical object of the present invention is to provide a polymer compound capable of selectively recognizing cancer cells including sialic acid.

Still another technical object of the present invention is to provide a polymer compound having an improved killing efficiency for cancer cells with sialic acid overexpressed thereon.

The technical objects of the present invention are not limited to the above-described objects.

### Technical Solution

To solve the technical problems described above, the present invention may provide a polymer compound.

According to one embodiment, there may be provided a polymer compound including a hydrophobic moiety binding to a natural killer (NK) cell, a cancer cell recognition moiety, and a linker, in which the hydrophobic moiety is bound to one end of the linker and the cancer cell recognition moiety is bound to the other end of the linker, so as to recognize NK cells and cancer cells, in which the cancer cell recognition moiety selectively recognizes solid cancer cells including sialic acid.

According to one embodiment, the cancer cell recognition moiety may include phenylboronic acid.

According to one embodiment, the cancer cell recognition moiety may bind to the sialic acid of the solid cancer cell while forming a boron-ester complex.

According to one embodiment, the polymer compound bound to the NK cell through the hydrophobic moiety may bind to cancer cells through the cancer cell recognition moiety, and then promote a secretion of cytotoxic granules and cytokines from the NK cells to kill the cancer cells.

According to one embodiment, the hydrophobic moiety may include a lipid, and may be bound to a surface of the NK cell through a hydrophobic interaction via the lipid.

According to one embodiment, the hydrophobic moiety may include any one of a phospholipid having an alkyl chain having 12 to 24 carbon atoms, a sterols lipid having 10 to 30 carbon atoms, 1,2-distearoyl-sn-glycero-3-phosphorylethanolamine (DSPE), 1,2-bis(diphenylphosphino)ethane (DPPE), and 1,2-bis(dimethylphosphino)ethane (DMPE).

According to one embodiment, the hydrophobic moiety may further include a sub-linker, in which the sub-linker is connected to one end of the lipid, and the hydrophobic moiety is bound to the linker through the sub-linker.

According to one embodiment, the linker may prevent the polymer compound bound to the NK cell from being subjected to endocytosis into the NK cell.

According to one embodiment, the linker may include polyethylene glycol (PEG).

According to one embodiment, the polymer compound may be represented by <Formula 1> below:

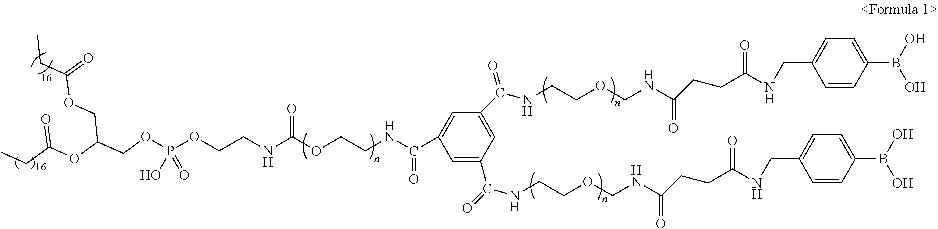

(n: an integer of 0 or more)

According to one embodiment, the cancer cell recognition moiety may recognize colorectal cancer cells (HCT-116), triple negative breast cancer cells (MDA-MB-231), and liver cancer cells (HepG2).

To solve the technical problems described above, the present invention may provide a method for preparing a polymer compound.

According to one embodiment, the method for preparing the polymer compound may include: mixing a hydrophobic moiety and a linker including an amine group at an end thereof to form a first intermediate structure in which the linker is connected to one end of the hydrophobic moiety and an amine group is included at an end thereof; preparing a second intermediate structure by converting an amine group of the first intermediate structure to a carboxylic acid group; and reacting the second intermediate structure with phenylboronic acid to prepare a polymer compound in which the hydrophobic moiety is bound to one end of the linker and the phenylboronic acid is bound to the other end thereof.

According to one embodiment, the second intermediate structure may have an amine group converted to a carboxylic acid group through succinylation of the first intermediate structure.

According to one embodiment, the hydrophobic moiety may include a compound in which polyethylene glycol (PEG) is bound to one end of 1,2-distearoyl-sn-glycero-3-phosphatidylethanolamine (DSPE), and the linker includes polyethylene glycol (PEG).

### Advantageous Effects

A polymer compound including a hydrophobic moiety (lipid), a cancer cell recognition moiety (phenylboronic acid), and a linker (PEG) connecting the hydrophobic moiety and the cancer cell recognition moiety can easily modify a surface of NK cells by a hydrophobic interaction through the hydrophobic moiety (lipid) (modification such that the NK cell can recognize a specific cancer cell), can selectively recognize solid cancer cells (e.g., colorectal cancer cells, triple negative breast cancer cells, liver cancer cells, etc.) with sialic acid overexpressed thereon among various cancer cells, and then can effectively kill the same.

### DESCRIPTION OF DRAWINGS

FIG. 1 is a view for describing a state in which a polymer compound according to an embodiment of the present invention is bound to an NK cell and a cancer killing process through the NK cell.

FIG. 2 is a view for describing a state in which a polymer compound according to an embodiment of the present invention is bound to an NK cell in more detail.

FIG. 3 is a view for describing a binding between a cancer cell recognition moiety of a polymer compound according to an embodiment of the present invention and sialic acid of cancer cells.

FIG. 4 is a view for describing S10 in a method for preparing a polymer compound according to an embodiment of the present invention.

FIG. 5 is a view for describing S20 in a method for preparing a polymer compound according to an embodiment of the present invention.

FIG. 6 is a view for describing S30 in a method for preparing a polymer compound according to an embodiment of the present invention.

FIGS. 7 and 8 are views showing FT-IR and 1H-NMR analysis results for a polymer compound according to an experimental example of the present invention, a compound used in a preparation process thereof, and products thereof.

FIG. 9 is a view for describing a binding of PBA, DSPE_{PEG}-di(PEG), and DSPE_{PEG}-di(PEG-PBA) to sialic acid.

FIG. 10 is a view for describing a binding ability between a polymer compound according to an experimental embodiment of the present invention and various sugars.

FIG. 11 shows an image of measured fluorescence intensity of cells in which a polymer compound according to an experimental example of the present invention is bound to NK cells.

FIG. 12 is a view for describing an NK cell-binding efficiency according to a concentration of a polymer compound according to an embodiment of the present invention.

FIG. 13 is a graph showing a viability of PBA-NK cell.

FIG. 14 is a graph for describing an effect of a polymer compound according to an experimental example of the present invention on cytokine secretion of NK cells.

FIG. 15 is a view for describing an effect of a polymer compound according to an experimental example of the present invention on a ligand of NK cells.

FIG. 16 is a view for describing an ability of PBA-NK cell to target cancer cells.

FIGS. 17 and 18 are views for describing an ability of PBA-NK cell to secrete granzyme B (perforin).

FIG. 19 is a view for describing an ability of PBA-NK cell to secrete cytokines (IFN-Y).

FIG. 20 is a view for describing an ability of PBA-NK cell to kill target cells.

FIG. 21 shows a fluorescence microscope image of a spheroid of triple negative breast cancer cells (MDA-MB-231) treated with NK cell and PBA-NK cell.

FIG. 22 is a graph quantifying a fluorescence intensity measured in FIG. 21.

FIG. 23 shows a fluorescence microscope image of a spheroid of triple negative breast cancer cells (MDA-MB-231) stained with EthD-1 after NK cell and PBA-NK cell are treated.

FIG. 24 is a graph quantifying the intensity and spheroid area of EthD-1 measured in FIG. 23.

### MODE FOR INVENTION

Hereinafter, preferred embodiments of the present invention will be described in detail with reference to the accompanying drawings. However, the technical spirit of the present invention is not limited to the embodiments described herein and may be implemented in other forms. Rather, the embodiments introduced herein are provided so that the disclosed contents may be thorough and complete and the spirit of the present invention may be sufficiently conveyed to those skilled in the art.

In this specification, when a component is referred to as being on another component, it means that it may be formed directly on the other component or a third component may be interposed therebetween. In addition, in the drawings, the thicknesses of films and regions are exaggerated for effective description of the technical contents.

Furthermore, in various embodiments of the present specification, terms such as first, second, third, etc., are used to describe various components, but these components should not be limited by these terms. These terms have only been used to distinguish one component from another component. Accordingly, a component mentioned as a first component in one embodiment may be mentioned as a second component in another embodiment. Each embodiment described and exemplified herein includes a complementary embodiment thereof. In addition, in the present specification, “and/or” is used as a meaning including at least one of the components listed before and after.

In the specification, a singular expression includes a plural expression unless the context clearly indicates otherwise. In addition, terms such as “include,” “have” or the like are intended to designate the presence of features, numbers, steps, components, or combinations thereof described in the specification, and should not be understood to preclude the possibility of the presence or addition of one or more other features, numbers, steps, components, or combinations thereof. In addition, in the present specification, “connection” is used as a meaning including both indirectly connecting a plurality of components and directly connecting the plurality of components.

Furthermore, in the following description of the present invention, when it is determined that a detailed description of a related known function or configuration may unnecessarily obscure the subject matter of the present invention, the detailed description thereof will be omitted.

### Polymer Compound According to Embodiment

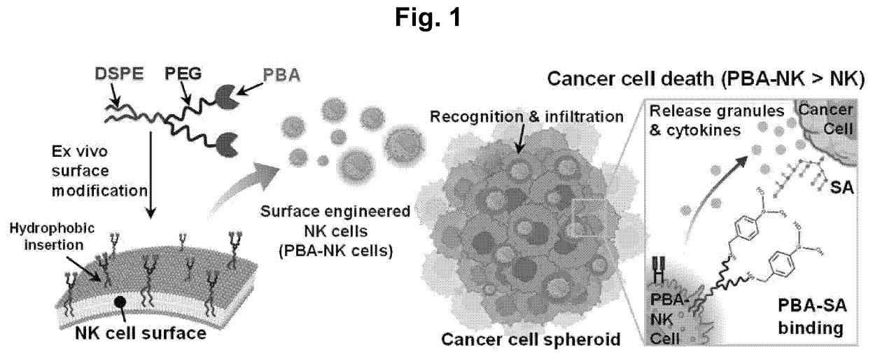

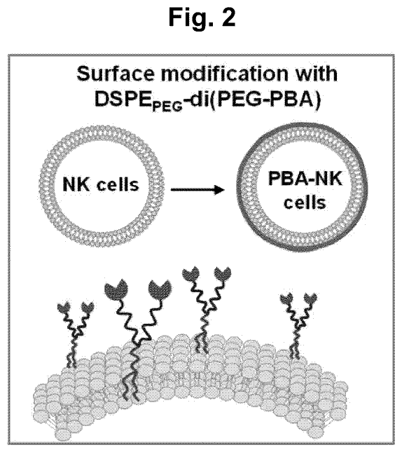

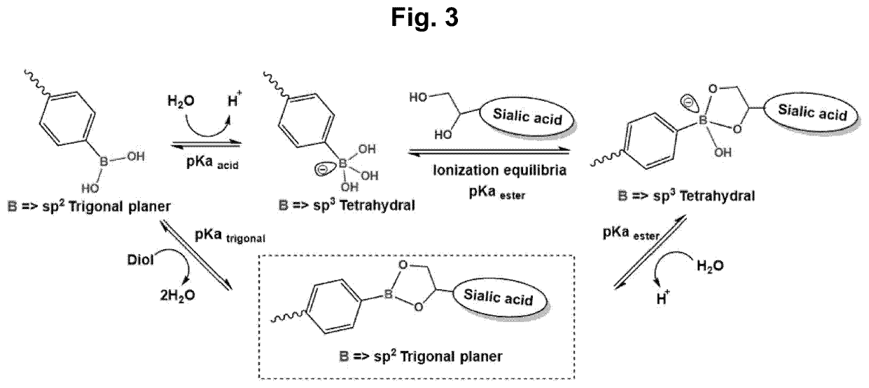

FIG. 1 is a view for describing a state in which a polymer compound according to an embodiment of the present invention is bound to an NK cell and a cancer killing process through the NK cell, FIG. 2 is a view for describing a state in which a polymer compound according to an embodiment of the present invention is bound to an NK cell in more detail, and FIG. 3 is a view for describing a bond between a cancer cell recognition moiety of a polymer compound according to an embodiment of the present invention and sialic acid of cancer cells.

Referring to FIGS. 1 and 3, the polymer compound according to an embodiment of the present invention may include a hydrophobic moiety, a cancer cell recognition moiety, and a linker connecting the hydrophobic moiety and the cancer cell recognition moiety. In other words, the polymer compound may have a structure in which the hydrophobic moiety is bound to one end of the linker and the cancer cell recognition moiety is bound to the other end thereof.

The hydrophobic moiety may recognize a natural killer (NK) cell and may be bound to a surface of the NK cell. Accordingly, the polymer compound may be immobilized on the surface of the NK cell by the hydrophobic moiety. More specifically, the hydrophobic moiety may be configured to include a lipid, and may be bound to the surface of the NK cell through a hydrophobic interaction via the lipid.

According to one embodiment, the hydrophobic moiety may include any one of a phospholipid having an alkyl chain having 12 to 24 carbon atoms, a sterols lipid having 10 to 30 carbon atoms, 1,2-distearoyl-sn-glycero-3-phosphorylethanolamine (DSPE), 1,2-bis(diphenylphosphino)ethane (DPPE), and 1,2-bis(dimethylphosphino)ethane (DMPE).

The hydrophobic moiety may further include a sub-linker, and may have a structure in which the sub-moiety is bound to one end of the lipid, in which the hydrophobic moiety is bound to the linker through the sub-linker. According to one embodiment, the sub-linker may include polyethylene glycol (PEG). For example, when DSPE is used as a lipid of the hydrophobic moiety and PEG is used as a sub-linker, the hydrophobic moiety may be represented by DSPE_{PEG}.

The cancer cell recognition moiety may recognize a cancer cell and bind to the cancer cell, and may include phenylboronic acid (PBA). As described above, when the cancer cell recognition moiety includes phenylboronic acid, the cancer cell recognition moiety may selectively recognize solid cancer cells including sialic acid. More specifically, the cancer cell recognition moiety including phenylboronic acid may selectively recognize cancer cells with sialic acid overexpressed thereon. For example, the cancer cells with sialic acid overexpressed thereon may include colorectal cancer cells (HCT-116), triple negative breast cancer cells (MDA-MB-231), and liver cancer cells (HepG2). In other words, the cancer cell recognition moiety including phenylboronic acid may selectively recognize colorectal cancer cells (HCT-116), triple negative breast cancer cells (MDA-MB-231), and liver cancer cells (HepG2). Accordingly, the NK cell bound to the hydrophobic moiety may selectively kill colorectal cancer cells (HCT-116), triple negative breast cancer cells (MDA-MB-231), and liver cancer cells (HepG2) among various cancer cells by the cancer cell recognition moiety.

The linker may be adapted for connecting the hydrophobic moiety and the cancer cell recognition moiety, and may include polyethylene glycol (PEG). In addition, the linker may prevent the polymer compound bound to the NK cell from being subjected to endocytosis into the NK cell.

As shown in FIG. 1, the NK killer cell bound to the polymer compound (PBA-NK cell) may recognize cancer cells including sialic acid through the cancer cell recognition moiety (phenylboronic acid, PBA) of the polymer compound, and then may be bound to cancer cells through the cancer cell recognition moiety (phenylboronic acid, PBA). More specifically, as shown in FIG. 3, the cancer cell recognition moiety (phenylboronic acid, PBA) may bind to the sialic acid of the solid cancer cell while forming a boron-ester complex. After that, an activation process in which cytotoxic granules and cytokines are secreted from the NK cell may be performed, and cancer cells may be killed by the cytotoxic granules and cytokines secreted from the NK cell. According to one embodiment, the polymer compound may promote the secretion of cytotoxic granules and cytokines from the NK cell during the activation process, thereby improving cancer cell killing efficiency.

According to one embodiment, the polymer compound may be represented by <Formula 1> below: In <Formula 1> below, a red structural formula may refer to the hydrophobic moiety (DSPE_{PEG}), a blue structural formula may mean the linker (PEG), and a green structural formula may represent the cancer cell recognition moiety (PBA). In other words, the polymer compound may have a structure in which two linkers (PEG) are bound to one end of the hydrophobic moiety (DSPE_{PEG}), and the cancer cell recognition moiety (PBA) is bound to each linker (PEG).

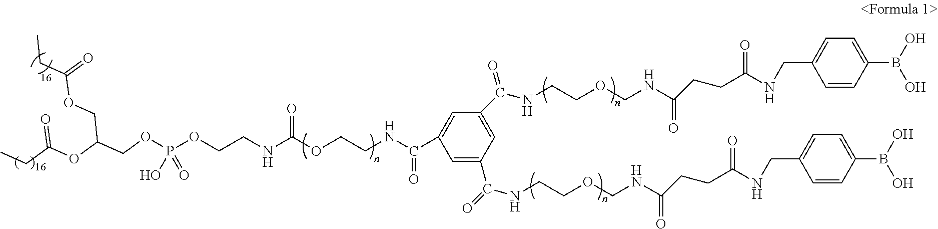

(n: an integer of 0 or more)

As a result, the polymer compound including a hydrophobic moiety (lipid), a cancer cell recognition moiety (phenylboronic acid), and a linker (PEG) connecting the hydrophobic moiety and the cancer cell recognition moiety may easily modify a surface of NK cells by a hydrophobic interaction through the hydrophobic moiety (lipid) (modification such that the NK cell may recognize a specific cancer cell), may selectively recognize solid cancer cells (e.g., colorectal cancer cells, triple negative breast cancer cells, liver cancer cells, etc.) with sialic acid overexpressed thereon among various cancer cells, and then may effectively kill the same.

### Method for Preparing Polymer Compound According to Embodiment

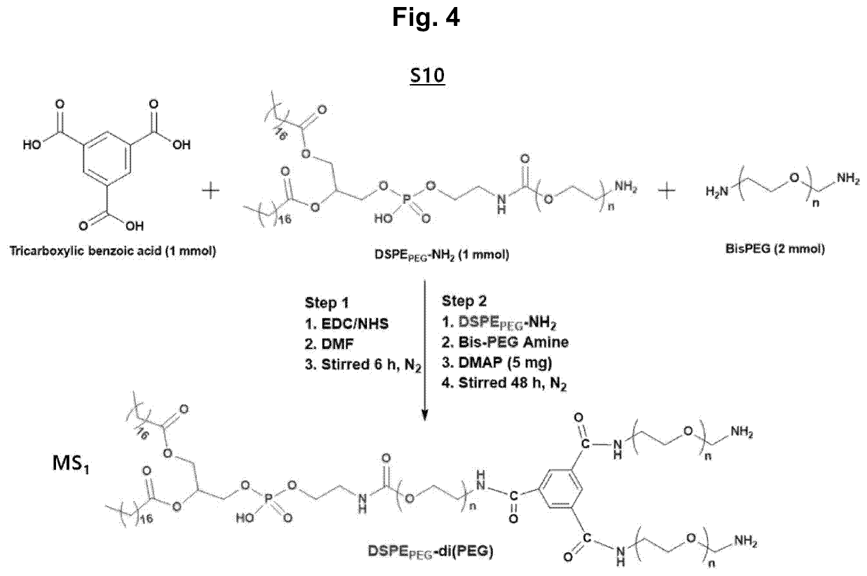

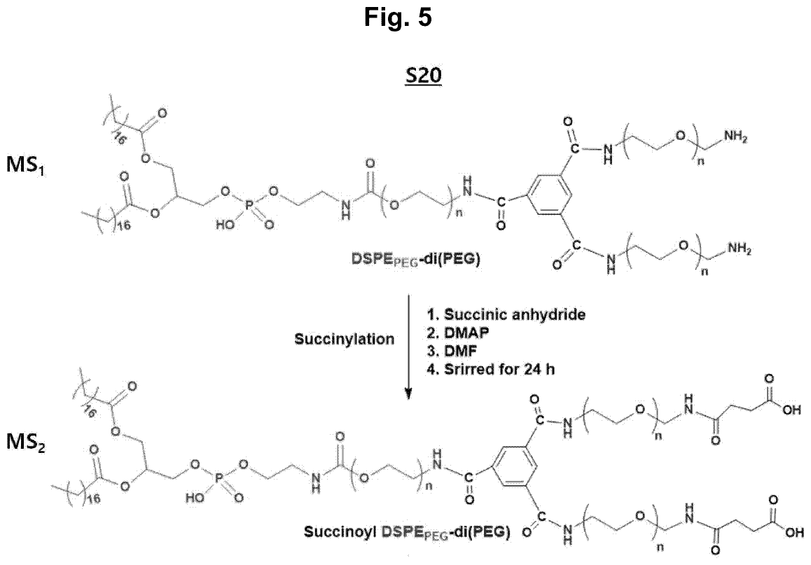

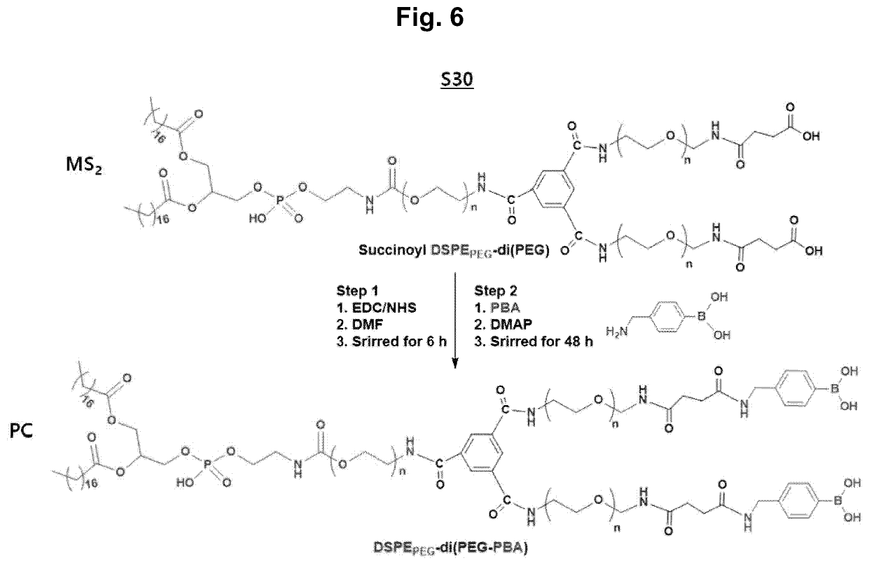

FIG. 4 is a view for describing S10 in a method for preparing a polymer compound according to an embodiment of the present invention, FIG. 5 is a view for describing S20 in a method for preparing a polymer compound according to an embodiment of the present invention, and FIG. 6 is a view for describing S30 in a method for preparing a polymer compound according to an embodiment of the present invention.

Referring to FIGS. 4 to 6, the method for preparing the polymer compound according to an embodiment of the present invention may include forming a first intermediate structure MS₁ (S10), forming a second intermediate structure MS₂, and forming a polymer compound PC (S30). Hereinafter, each step will be described in detail.

In above S10, the first intermediate structure MS₁ may be formed by mixing tricarboxylic benzoic acid, a hydrophobic moiety, and a linker. According to one embodiment, the hydrophobic moiety may include a compound (DSPE_{PEG}-NH₂) in which a sub-linker (PEG) is bound to one end of the lipid (DSPE) and an amine group (NH₂) is provided at an end of the sub-linker (PEG). According to one embodiment, the linker may include a compound (Bis-PEG Amine) having an amine group (NH₂) at an end of PEG. In one embodiment, the first intermediate structure MS₁ may include a compound (DSPE_{PEG}-di(PEG)) in which the two linkers are connected to one end of the hydrophobic moiety and an amine group is provided at an end thereof. A structural formula of the compound used as the hydrophobic moiety may be the same as DSPE_{PEG}-NH₂ shown in FIG. 4, a structural formula of the compound used as the linker may be the same as BisPEG shown in FIG. 4, and a structural formula of the compound represented by the first intermediate structure MS₁ may be the same as DSPE_{PEG}-di(PEG) shown in FIG. 4.

In above S20, a second intermediate structure MS₂ may be formed by converting an amine group at an end of the first intermediate structure MS₁ into a carboxylic acid group through the succinylation of the first intermediate structure MS₁. A structural formula of the compound represented by the second intermediate structure MS₂ may be the same as succinoyl DSPE PEG-di(PEG) shown in FIG. 5.

In above S30, the second intermediate structure MS₂ and phenylboronic acid (PBA) may be reacted to prepare a polymer compound (PC) in which the hydrophobic moiety (DSPE_{PEG}) is bound to one end of the linker (PEG) and the phenylboronic acid (PBA) is bound to the other end thereof. The structural formula of the polymer compound (PC) may be the same as DSPE_{PEG}-di(PEG-PBA) shown in FIG. 6.

Hereinafter, the polymer compound according to an embodiment of the present invention will be described in more detail through specific experimental examples.

### Preparation of Polymer Compound (DSPE_{PEG}-Di(PEG-PBA)) According to Experimental Embodiment

1 mmol of tricarboxylic benzoic acid, 3 mmol of 1-ethyl-3-(3-dimethylaminopropyl) carbodiimide (EDC), and 5 mmol of N-hydroxy succinimide (NHS, Sigma-Aldrich) were dissolved in 5 mL of dimethyl formamide (DMF), and then stirred at room temperature (RT) under an N₂ atmosphere to prepare a first base solution.

2 mmol of Bis-PEG Amine, 1 mmol of DSPE_{PEG}-NH₂, and 5 mg of 4-dimethyl amino pyridine (DMAP) were added to the first base solution prepared and stirred at room temperature (RT) under an N₂ atmosphere for 48 hours. The mixture obtained through stirring was transferred to a dialysis tube (MWCO 6 kDa) and dialyzed with distilled water (DW) for three days to remove unreacted materials, and then freeze-dried to prepare the first intermediate structure (DSPE_{PEG}-di(PEG)).

The first intermediate structure (DSPE_{PEG}-di(PEG)), an excess of succinic anhydride, and DMAP were dissolved in anhydrous DMF, and then stirred at room temperature (RT) under an N₂ atmosphere for 24 hours. The mixture obtained through stirring was transferred to a dialysis tube (MWCO 2 kDa) and dialyzed with distilled water (DW) for three days to remove unreacted materials, and then freeze-dried to prepare the second intermediate structure (succinoyl DSPE_{PEG}-di(PEG)).

1 mmol of the second intermediate structure (succinoyl DSPE_{PEG}-di(PEG)), an excess of EDC, and an excess of NHS were dissolved in 5 mL of anhydrous DMF, and then stirred at room temperature (RT) for 48 hours to prepare a second base solution.

5 mmol of phenylboronic acid (PBA) and 5 mg of DMAP were added to the second base solution and stirred at room temperature (RT) under an N₂ atmosphere for 48 hours. The mixture obtained through stirring was transferred to a dialysis tube (MWCO 2 kDa) and dialyzed with distilled water (DW) for three days to remove unreacted materials, and then freeze-dried to prepare DSPE_{PEG}-di(PEG-PBA) polymer compound. The preparation method described above are shown in FIGS. 4 to 6.

### Experimental Example 1: Characterization of Polymer Compound

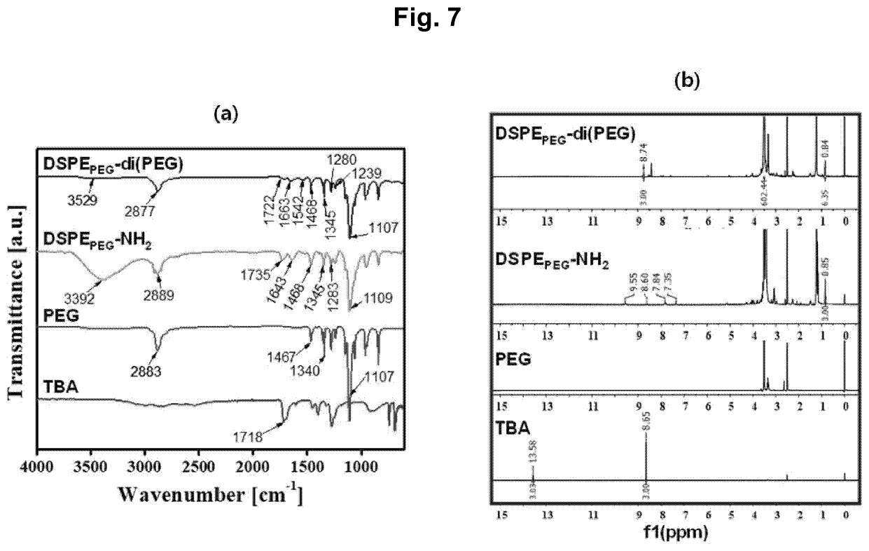

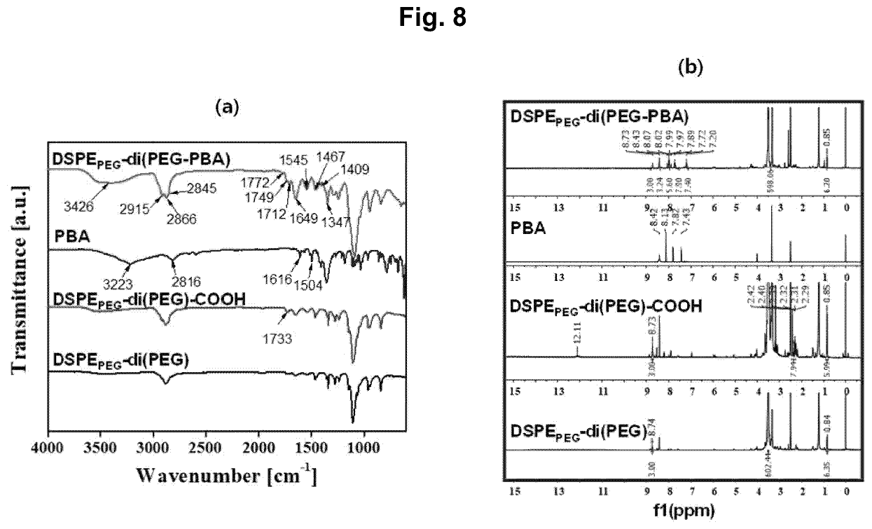

FIGS. 7 and 8 are views showing FT-IR and 1H-NMR analysis results for a polymer compound according to an experimental example of the present invention, a compound used in a preparation process thereof, and products thereof.

Referring to FIG. 7, it may show FT-IR analysis results (a) and 1H-NMR analysis results (b) for each of tricarboxylic benzoic acid (TBA), polyethylene glycol (PEG), and DSPE_{PEG}-NH₂ used in the preparation process of the DSPE_{PEG}-di(PEG-PBA) polymer compound according to the experimental example, and DSPE_{PEG}-di(PEG), which is a first intermediate structure generated in the preparation process. As can be seen from FIG. 7, it can be confirmed that DSPE_{PEG}-di(PEG), which is the first intermediate structure, was synthesized through the use of tricarboxylic benzoic acid (TBA), polyethylene glycol (PEG), and DSPE_{PEG}-NH₂.

Referring to FIG. 8, it may show FT-IR analysis results (a) and ¹H-NMR analysis results (b) for each of phenylboronic acid (PBA) used in the preparation process of the DSPE_{PEG}-di(PEG-PBA) polymer compound according to the experimental example, DSPE_{PEG}-di(PEG), which is the first intermediate structure generated in the preparation process, DSPE_{PEG}-di(PEG)-COOH, which is a second intermediate structure, and DSPE_{PEG}-di(PEG-PBA), which is a final product. As can be seen from FIG. 8, it can be confirmed that phenylboronic acid (PBA) was bound to DSPE_{PEG}-di(PEG)-COOH, which is the second intermediate structure, to synthesize DSPE_{PEG}-di(PEG-PBA), which is the final product.

### Experimental Example 2: Analysis of Binding Between Polymer Compound and Sugar

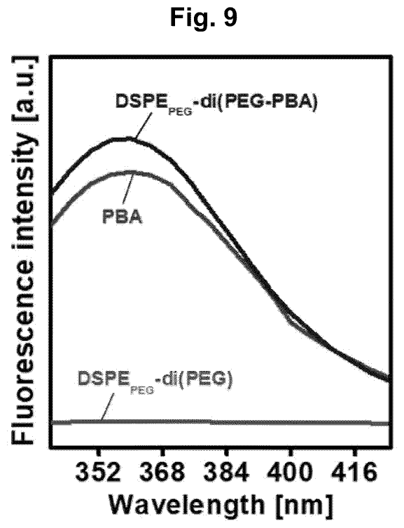

FIG. 9 is a view for describing a binding of PBA, DSPE_{PEG}-di(PEG), and DSPE_{PEG}-di(PEG-PBA) to sialic acid.

Referring to FIG. 9, it may show fluorescence intensity (a.u.) measured from a mixture obtained by mixing each of PBA, DSPE_{PEG}-di(PEG), and DSPE_{PEG}-di(PEG-PBA) with a pH 7.4 buffer solution and sialic acid. When PBA is bound to sugar, it may exhibit a fluorescent photo-induced electron transfer (PET) effect, thus showing a presence of binding between the compounds described above and sialic acid through the measurement of fluorescence intensity.

As can be seen from FIG. 9, it can be confirmed that in the case of DSPE_{PEG}-di(PEG) without PBA, fluorescence intensity was not measured, whereas in the case of PBA and DSPE_{PEG}-di(PEG-PBA), fluorescence intensity was measured. In other words, it can be seen that DSPE_{PEG}-di(PEG) without PBA is not bound to sialic acid, whereas PBA and DSPE_{PEG}-di(PEG-PBA) are bound to sialic acid.

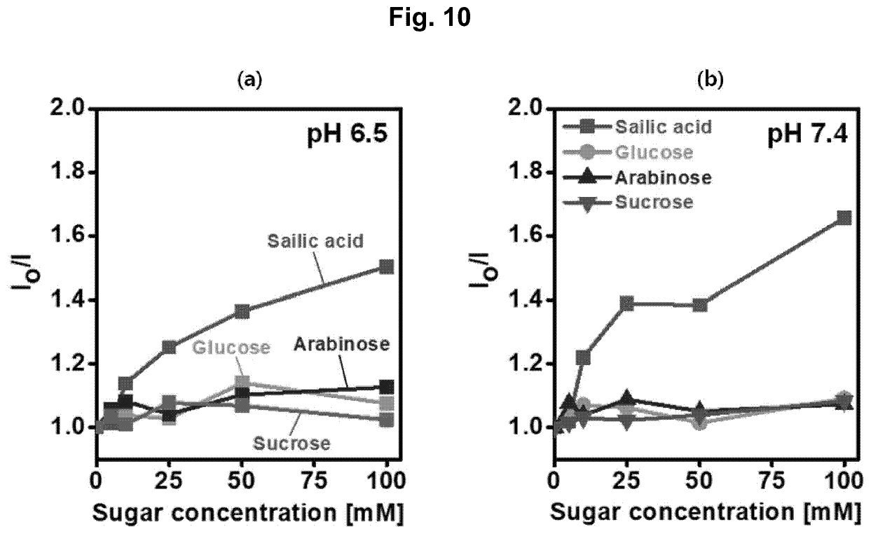

FIG. 10 is a view for describing a binding ability between a polymer compound according to an experimental embodiment of the present invention and various sugars.

Referring to FIG. 10, it may show the results obtained by mixing DSPE_{PEG}-di(PEG-PBA) with a buffer solution and various sugars, and then analyzing fluorescence intensity measured from the mixture to confirm binding affinity. More specifically, sialic acid, glucose, arabinose, and sucrose were used as sugars, and the binding affinity was confirmed as a value (Iₒ/I) of a fluorescence intensity (I) compared to an initial fluorescence intensity (Iₒ). In addition, (a) of FIG. 10 may show the result of using a buffer solution of pH 6.5 as a buffer solution, and (b) of FIG. 10 may show the result of using a buffer solution of pH 7.4 as a buffer solution.

As can be seen from FIG. 10, it can be confirmed that DSPE PEG-di(PEG-PBA), which is the polymer compound according to the experimental example, has a significantly higher binding affinity with sialic acid compared to other sugars (glucose, arabinose, sucrose).

### Experimental Example 3: Analysis of Fluorescence Intensity of NK Cell Bound to Polymer Compound

A polymer compound (DSPE_{PEG}-di(PEG-5CFL)) was dissolved in MEM alpha at a concentration of 1.0 mg/mL, and 5×10⁵ NK92-mi NK cells were evenly mixed with 100 μL of solution. The surface of NK cell was modified (DSPE_{PEG}-di(PEG-5CFL) bound to NK cell) at room temperature for 30 minutes and then washed twice with MEM alpha. After that, cells were lysed with 250 μL of RIPA buffer and stored at 4° C. for 30 minutes. Finally, 250 μL of distilled water was added, diluted, and transferred to 96-well to measure fluorescence intensity at 480/535 nm. 5-carboxyfluorescein (5CFL), which is a fluorescent dye, was used instead of PBA to measure fluorescence intensity.

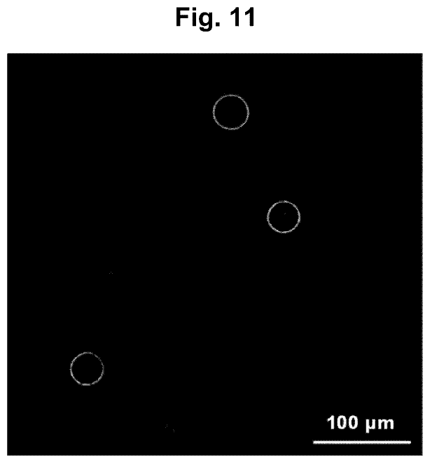

FIG. 11 shows an image of measured fluorescence intensity of cells in which a polymer compound according to an experimental example of the present invention is bound to NK cells.

Referring to FIG. 11, it may show an image of measured fluorescence intensity of the cells in which DSPE_{PEG}-di(PEG-5CFL) is bound to NK cell as described in Experimental Example 3. As can be seen from FIG. 11, it can be confirmed that DSPE_{PEG}-di(PEG-5CFL) is evenly coated on a surface of NK cells.

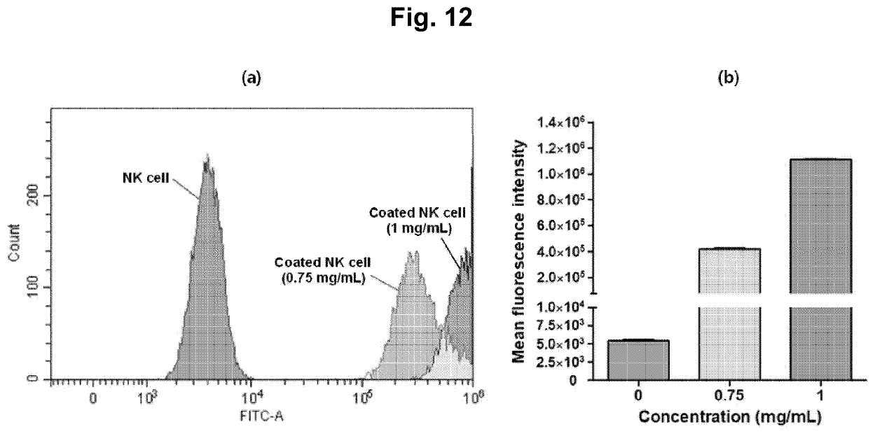

FIG. 12 is a view for describing an NK cell-binding efficiency according to a concentration of a polymer compound according to an embodiment of the present invention.

Referring to FIG. 12, it may quantitatively show a result of measuring the fluorescence intensity for each of NK cell, coated NK cell (0.75 mg/mL), and coated NK cell (1 mg/mL) by the method described through Experimental Example 3. More specifically, NK cell may refer to a state in which DSPE_{PEG}-di(PEG-5CFL) is not bound, coated NK cell (0.75 mg/mL) may represent a state in which 0.75 mg/mL of DSPE_{PEG}-di(PEG-5CFL) is bound to NK cell, and coated NK cell (1 mg/mL) may mean a state in which 1 mg/mL of DSPE_{PEG}-di(PEG-5CFL) is bound to NK cell.

As can be seen from FIG. 12, it can be confirmed that the NK cell to which DSPE_{PEG}-di(PEG-5CFL) is bound shows a higher fluorescence intensity than that of NK cell to which DSPE_{PEG}-di(PEG-5CFL) is not bound, and the NK cell to which 1 mg/mL of DSPE_{PEG}-di(PEG-5CFL) is bound shows a higher fluorescence intensity than that of 0.75 mg/mL of DSPE_{PEG}-di(PEG-5CFL). In other words, it can be seen that 1 mg/mL of DSPE_{PEG}-di(PEG-PBA) is suitable for surface modification of NK cell.

### Experimental Example 4: Analysis of Effect of Polymer Compound on NK Cell

A polymer compound (DSPE_{PEG}-di(PEG-PBA)) was dissolved in MEM alpha at a concentration of 1.0 mg/mL, and 5×10⁵ NK92-mi NK cells were evenly mixed with 100 μL of solution. The surface of NK cell was modified (DSPE_{PEG}-di(PEG-PBA) bound to NK cell) at room temperature for 30 minutes and then washed twice with MEM alpha. After that, cells were lysed with 250 μL of RIPA buffer and stored at 4° C. for 30 minutes. A cell in which DSPE_{PEG}-di(PEG-PBA) is bound to NK cell is expressed as PBA-NK cell.

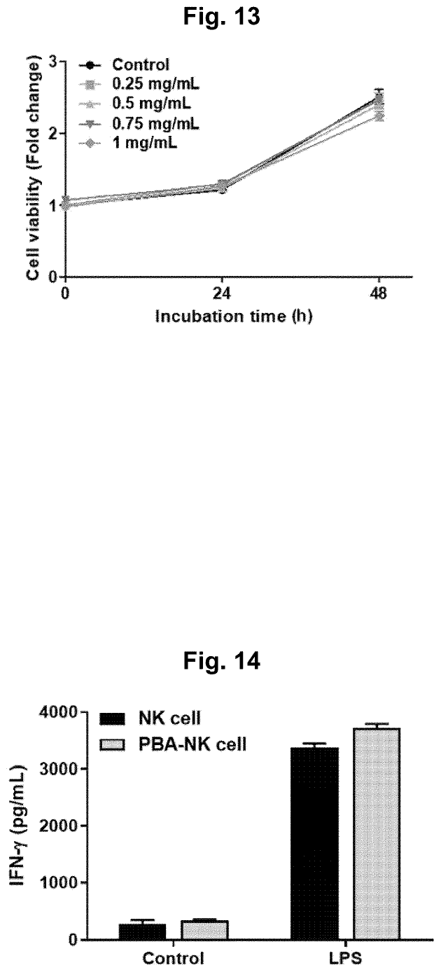

FIG. 13 is a graph showing a viability of PBA-NK cell.

Referring to FIG. 13, it may show the viability (fold change) of NK cell (PBA-NK cell) bound to DSPE_{PEG}-di(PEG-PBA) at various concentrations. More specifically, it may show a result of using 0.25 mg/mL, 0.5 mg/mL, 0.75 mg/mL, and 1 mg/mL of DSPE_{PEG}-di(PEG-PBA). In addition, it may show the viability of NK cell to which DSPE_{PEG}-di(PEG-PBA) is not bound as a control.

As can be seen from FIG. 13, it can be confirmed that NK cell (PBA-NK cell) bound to DSPE_{PEG}-di(PEG-PBA) has no substantial difference in viability from NK cell to which DSPE_{PEG}-di(PEG-PBA) is not bound. In other words, it can be confirmed that DSPE PEG-di(PEG-PBA) does not substantially affect the viability of NK cell. In addition, it can be confirmed that the concentration of DSPE_{PEG}-di(PEG-PBA) does not affect viability.

FIG. 14 is a graph for describing an effect of a polymer compound according to an experimental example of the present invention on cytokine secretion of NK cells.

Referring to FIG. 14, in order to evaluate whether the secretion of cytokine (IFN-Y), which is a representative material of NK cell for cancer cell death, normally functions, it may show a result obtained by quantifying an amount (pg/mL) of cytokine (IFN-Y) secreted from NK cell and PBA-NK cell. (a) of FIG. 14 may show a state in which LPS, which is a substance for promoting cytokine secretion, is not treated, and (b) of FIG. 14 may show a state in which LPS is treated.

As can be seen from FIG. 14, it can be confirmed that the amounts of cytokines (IFN-Y) secretion from NK cell and PBA-NK cell are similar to each other. In other words, when DSPE_{PEG}-di(PEG-PBA) is bound to NK cell, it can be confirmed that this case does not affect cytokine (IFN-Y) secretion, which is one of the unique functions of NK cell.

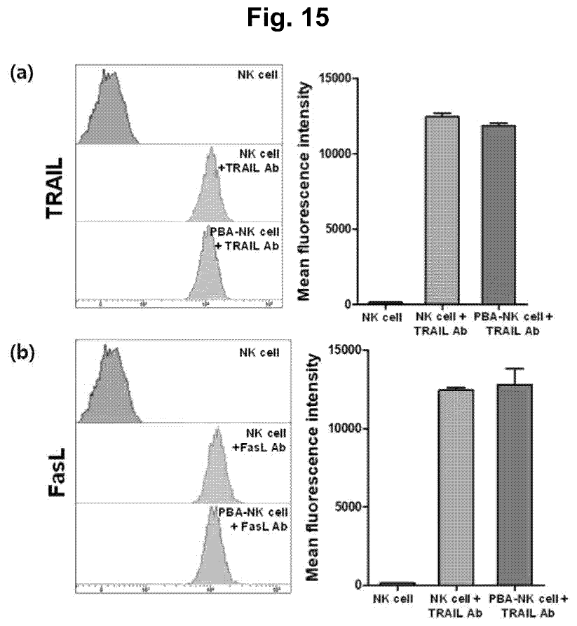

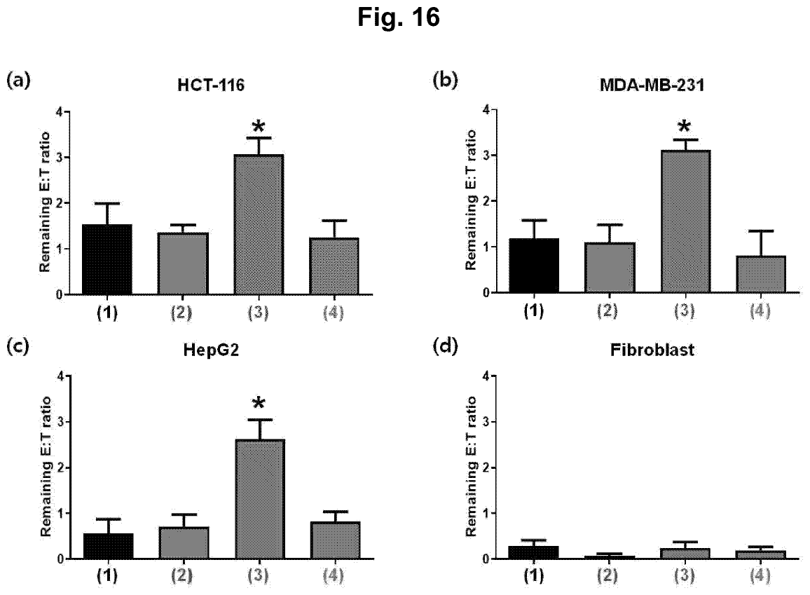

FIG. 15 is a view for describing an effect of a polymer compound according to an experimental example of the present invention on a ligand of NK cells.

Referring to FIG. 15, in order to evaluate whether TRAIL and FasL, which are two representative cell membrane ligands required for cancer cell recognition by NK cell, function normally, it may show the result obtained by making an MFI analysis by flow cytometry after treating NK cell and PBA-NK cell with TRAIL antibody and FasL antibody, and then detecting TRAIL and FasL present on the surface of each cell. (a) of FIG. 15 may show the results for TRAIL, and (b) of FIG. 15 may show the results for FasL.

As can be seen from FIG. 15, it can be confirmed that TRAIL and FasL ligands normally function in both NK cell and PBA-NK cell. In other words, when DSPE_{PEG}-di(PEG-PBA) is bound to NK cell, it can be confirmed that this case does not affect TRAIL and FasL ligands, which are unique ligands of NK cell.

### Experimental Example 5: Analysis of Cancer Cell Targeting Ability of PBA-NK Cell

NK cell was stained with calcein AM (green reagent) and target cell was stained with cell tracker red (red reagent), which were then bound to DSPE_{PEG}-di(PEG-PBA). After that, the bound cells were co-incubated with target cell at a ratio of 1:1 for 30 minutes, after which an effector cell cluster and a target cell cluster (E:T cluster) caught in both FITC and APC regions were detected by flow cytometry.

FIG. 16 is a view for describing an ability of PBA-NK cell to target cancer cells.

Referring to FIG. 16, E:T cluster was detected by the method according to Example 5 described above, but colorectal cancer cells (HCT-116), triple negative breast cancer cells (MDA-MB-231), liver cancer cells (HepG2) with sialic acid overexpressed thereon, and fibroblasts, which are normal cells, were used as the target cell. In addition, (1) in (a) to (d) of FIG. 16 may show a result for target cell in NK cell to which DSPE_{PEG}-di(PEG-PBA) is not bound, (2) may indicate a result for SA_{block} target cell in NK cell to which DSPE_{PEG}-di(PEG-PBA) is not bound, (3) may suggest a result for target cell in PBA-NK cell to which DSPE_{PEG}-di(PEG-PBA) is bound to NK cell, and (4) may show a result for SA_{block} target cell in PBA-NK cell to which DSPE_{PEG}-di(PEG-PBA) is bound to NK cell. In addition, SA_{block} target cell may refer to a target cell in which sialic acid is blocked by treating the target cell with an excessive amount of PBA.

As can be seen from (a) to (c) of FIG. 16, it can be confirmed that PBA-NK cell has a significantly higher targeting ability for colorectal cancer cells (HCT-116), triple negative breast cancer cells (MDA-MB-231), and liver cancer cells (HepG2) compared to NK cell. In addition, it can be confirmed that the targeting ability of PBA-NK cell for SA_{block} Target cell is significantly low. In addition, as can be seen from (d) of FIG. 16, it can be confirmed that PBA-NK cell has substantially no targeting ability for fibroblasts, which are normal cells. Accordingly, it can be seen that DSPE_{PEG}-di(PEG-PBA) may selectively recognize cancer cells (colorectal cancer cells, triple negative breast cancer cells, liver cancer cells) with sialic acid overexpressed thereon.

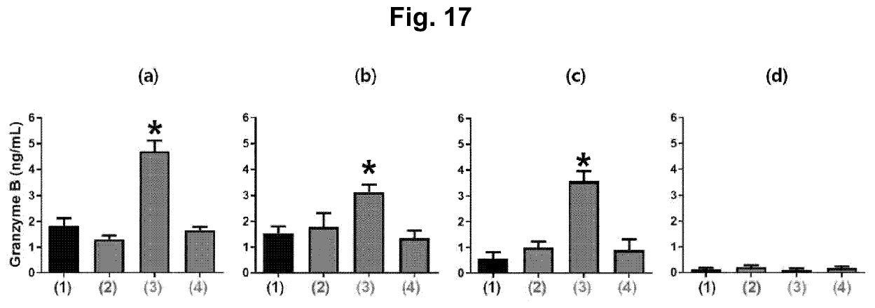

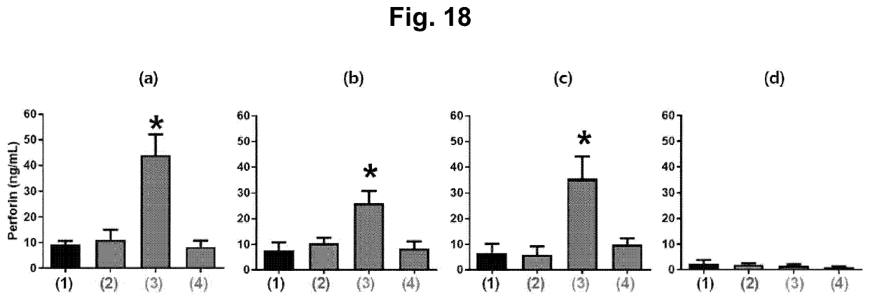

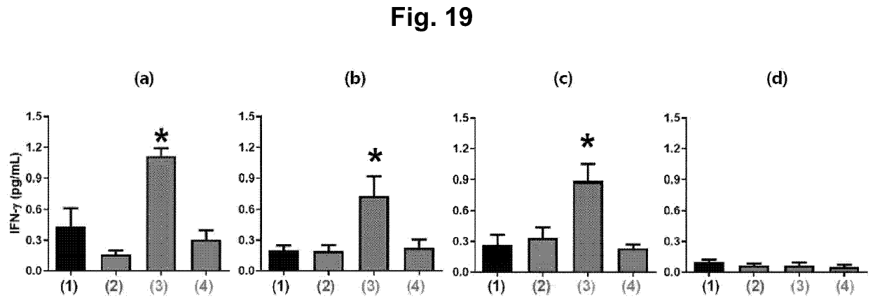

FIGS. 17 and 18 are views for describing an ability of PBA-NK cell to secrete granzyme B (perforin), and FIG. 19 is a view for describing an ability of PBA-NK cell to secrete cytokines (IFN-Y).

Referring to FIG. 17, it may show a measured amount of granzyme B secretion (ng/ml) of NK cell and PBA-NK cell to target cell. Referring to FIG. 18, it may show a measured amount of perforin secretion (ng/ml) of NK cell and PBA-NK cell to target cell. And, referring to FIG. 19, it may show a measured amount of IFN-Y secretion (ng/ml) of NK cell and PBA-NK cell to target cell. The target cell used herein may include colorectal cancer cells (HCT-116), triple negative breast cancer cells (MDA-MB-231), liver cancer cells (HepG2) with sialic acid overexpressed thereon, and fibroblasts, which are normal cells. In addition, (1) in (a) to (d) of FIGS. 17 to 19 may refer to a result for target cell in NK cell, (2) may represent a result for SA_{block} target cell in NK cell, (3) may indicate a result for target cell in PBA-NK cell, and (4) may refer to a result for SA_{block} target cell in PBA-NK cell. In addition, SA_{block} target cell may refer to a target cell in which sialic acid is blocked by treating the target cell with an excessive amount of PBA.

As can be seen from FIGS. 17 to 19, it can be confirmed that PBA-NK cell has a significantly higher amount of cytotoxic granule (granzyme B, perforin) and cytokine (IFN-Y) secretion for target cell compared to NK cell. In other words, it can be seen that DSPE_{PEG}-di(PEG-PBA) promotes the secretion of cytotoxic granules (granzyme B, perforin) and cytokine (IFN-Y) from NK cell. In addition, it can be confirmed that an amount of cytotoxic granules (granzyme B, perforin) secretion and an amount of cytokine (IFN-Y) secretion by PBA-NK cell for SA_{block} target cell is significantly low, and the cytotoxic granules (granzyme B, perforin) and cytokine (IFN-Y) are not substantially secreted for fibroblasts, which are normal cells. Accordingly, it can be seen that DSPE_{PEG}-di(PEG-PBA) may selectively recognize cancer cells (colorectal cancer cells, triple negative breast cancer cells, liver cancer cells) with sialic acid overexpressed thereon.

### Experimental Example 6: Analysis of Cancer Cell Targeting Ability of PBA-NK Cell

A DSPE_{PEG}-di(PEG-PBA) polymer compound was dissolved in MEM alpha at a concentration of 1.0 mg/mL, and 6×10⁵ NK92-mi NK cells were evenly mixed with 120 μL of composite material solution. The surface of NK cell was modified (HA-PEG-DSPE bound to NK cell) at room temperature for 30 minutes and then washed twice with MEM alpha.

6×10⁴ target cells were stained with 15 μM calcein-AM solution in an amount of 600 μL at 37° C. for 30 minutes, and then washed twice with HDMEM. The NK cell of which the surface is completely modified and the target cell which is completely stained were added together at a ratio of 1:1, 5:1 and 10:1 to 96-well plate and incubated at 37° C. for four hours, after which a supernatant was obtained and a fluorescence intensity was measured at 480/535 mm.

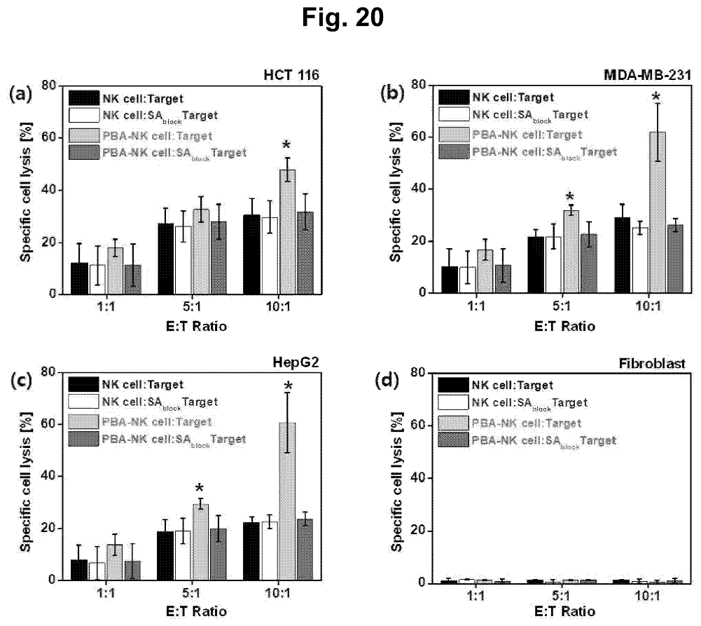

FIG. 20 is a view for describing an ability of PBA-NK cell to kill target cells.

Referring to FIG. 20, a solubility (specific cell lysis, %) of the target cell was measured by the method according to Experimental Example 6 described above, in which colorectal cancer cells (HCT-116), triple negative breast cancer cells (MDA-MB-231), liver cancer cells (HepG2) with sialic acid overexpressed thereon, and fibroblasts, which are normal cells, were used as the target cell. In addition, a black indicator in (a) to (d) of FIG. 20 may refer to a result for target cell in NK cell, a purple indicator may represent a result for SA_{block} target cell in NK cell, a green indicator may indicate a result for target cell in PBA-NK cell, and a red indicator may refer to a result for SA_{block} target cell in PBA-NK cell. In addition, SA_{block} target cell may refer to a target cell in which sialic acid is blocked by treating the target cell with an excessive amount of PBA.

As can be seen from FIG. 20, it can be confirmed that PBA-NK cell has a significantly higher solubility (specific cell lysis, %) for target cell compared to NK cell. In other words, it can be seen that DSPE_{PEG}-di(PEG-PBA) improves the ability of NK cell to kill colorectal cancer cells (HCT-116), triple negative breast cancer cells (MDA-MB-231), and liver cancer cells (HepG2). In addition, t can be seen that cell does not have a substantial ability to kill fibroblasts, which are normal cells. In other words, it can be seen that NK cell to which DSPE_{PEG}-di(PEG-PBA) is bound selectively kills cancer cells with sialic acid overexpressed thereon.

### Experimental Example 7: Analysis of Tumor Spheroid Destruction Ability of PBA-NK Cell

Spheroids of triple negative breast cancer cells (MDA-MB-231) were cultured with NK cell and PBA-NK cell, respectively, and then the ability to destroy the structure was measured by measuring fluorescence intensity.

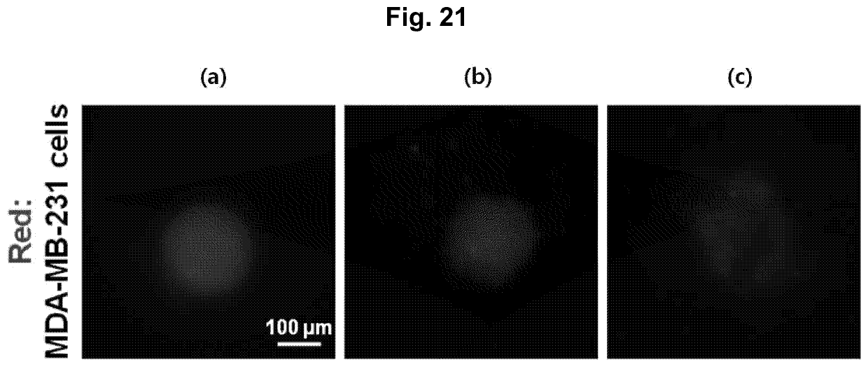

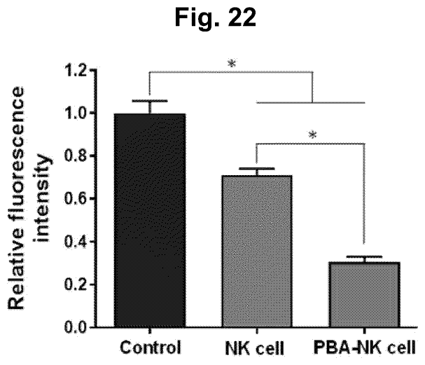

FIG. 21 shows a fluorescence microscope image of a spheroid of triple negative breast cancer cells (MDA-MB-231) treated with NK cell and PBA-NK cell, and FIG. 22 is a graph quantifying a fluorescence intensity measured in FIG. 21.

(a) of FIG. 21 may show a spheroid of triple negative breast cancer cells (MDA-MB-231) as a control, (b) may exhibit a spheroid of triple negative breast cancer cells (MDA-MB-231) treated with NK cell, and (c) may indicate a spheroid of triple negative breast cancer cells (MDA-MB-231) treated with PBA-NK cell.

As can be seen from FIGS. 21 and 22, it can be confirmed that PBA-NK cell has a significantly higher destruction ability for a spheroid of triple negative breast cancer cells (MDA-MB-231) compared to NK cell.

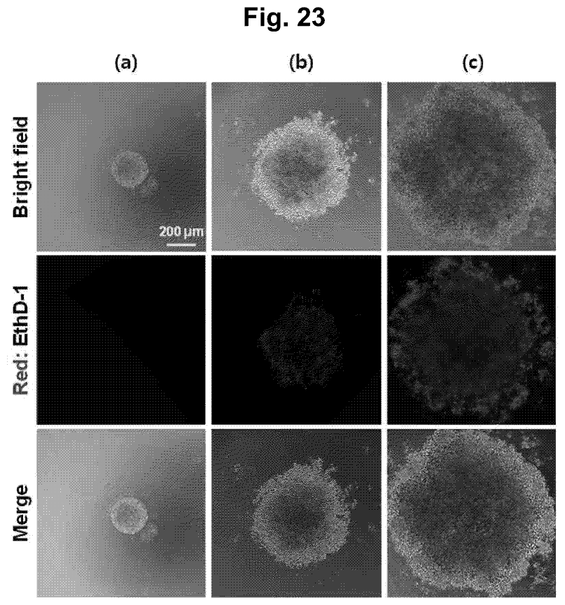

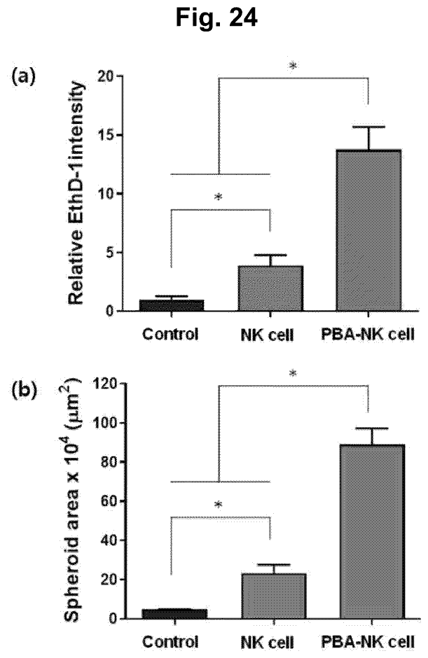

FIG. 23 shows a fluorescence microscope image of a spheroid of triple negative breast cancer cells (MDA-MB-231) stained with EthD-1 after NK cell and PBA-NK cell are treated, and FIG. 24 is a graph quantifying the intensity and spheroid area of EthD-1 measured in FIG. 23.

(a) of FIG. 23 may show a spheroid of triple negative breast cancer cells (MDA-MB-231) as a control, (b) may exhibit a spheroid of triple negative breast cancer cells (MDA-MB-231) treated with NK cell, and (c) may indicate a spheroid of triple negative breast cancer cells (MDA-MB-231) treated with PBA-NK cell.

As can be seen from FIGS. 23 and 24, it can be confirmed that PBA-NK cell has a significantly higher destruction ability for a spheroid of triple negative breast cancer cells (MDA-MB-231) compared to NK cell.

Although the present invention has been described in detail using preferred embodiments, the scope of the present invention is not limited to specific embodiments and should be interpreted by the appended claims. In addition, it should be understood by those skilled in the art that many modifications and variations are possible without departing from the scope of the present invention.

### INDUSTRIAL APPLICABILITY

The present invention may be used in the medical industry.

1. A polymer compound comprising a hydrophobic moiety binding to a natural killer (NK) cell, a cancer cell recognition moiety, and a linker, wherein
the hydrophobic moiety is bound to one end of the linker and the cancer cell recognition moiety is bound to an other end of the linker, so as to recognize NK cells and cancer cells, in which
the cancer cell recognition moiety selectively recognizes solid cancer cells including sialic acid.

2. The polymer compound of claim 1, wherein the cancer cell recognition moiety includes phenylboronic acid.

3. The polymer compound of claim 2, wherein the cancer cell recognition moiety binds to the sialic acid of the solid cancer cell while forming a boron-ester complex.

4. The polymer compound of claim 1, wherein the polymer compound bound to the NK cell through the hydrophobic moiety binds to the cancer cell through the cancer cell recognition moiety, and then
promotes a secretion of cytotoxic granules and cytokines from the NK cell to kill the cancer cell.

5. The polymer compound of claim 1, wherein the hydrophobic moiety includes a lipid, and is bound to a surface of the NK cell through a hydrophobic interaction via the lipid.

6. The polymer compound of claim 5, wherein the hydrophobic moiety includes any one of a phospholipid having an alkyl chain having 12 to 24 carbon atoms, a sterols lipid having 10 to 30 carbon atoms, 1,2-distearoyl-sn-glycero-3-phosphorylethanolamine (DSPE), 1,2-bis(diphenylphosphino)ethane (DPPE), and 1,2-bis(dimethylphosphino)ethane (DMPE).

7. The polymer compound of claim 5, wherein the hydrophobic moiety further includes a sub-linker, in which
the sub-linker is connected to one end of the lipid, and the hydrophobic moiety is bound to the linker through the sub-linker.

8. The polymer compound of claim 1, wherein the linker prevents the polymer compound bound to the NK cell from being subjected to endocytosis into the NK cell.

9. The polymer compound of claim 1, wherein the linker includes polyethylene glycol (PEG).

10. The polymer compound of claim 1, wherein the polymer compound is represented by: 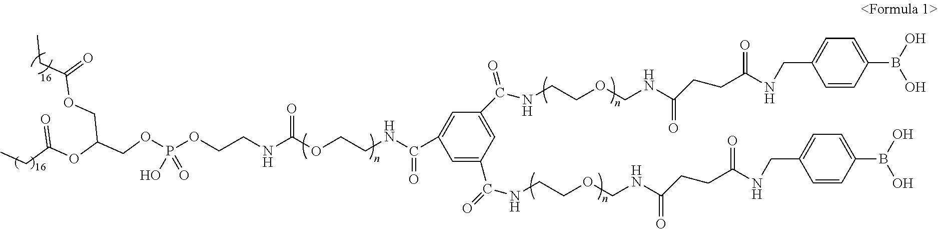
where n is an integer of 0 or more.

11. The polymer compound of claim 1, wherein the cancer cell recognition moiety recognizes colorectal cancer cells (HCT-116), triple negative breast cancer cells (MDA-MB-231), and liver cancer cells (HepG2).

12. A method for preparing a polymer compound, the method comprising:
mixing a hydrophobic moiety and a linker including an amine group at an end thereof to form a first intermediate structure in which the linker is connected to one end of the hydrophobic moiety and an amine group is included at an end thereof;
preparing a second intermediate structure by converting an amine group of the first intermediate structure to a carboxylic acid group; and
reacting the second intermediate structure with phenylboronic acid to prepare a polymer compound in which the hydrophobic moiety is bound to one end of the linker and the phenylboronic acid is bound to an other end thereof.

13. The polymer compound of claim 12, wherein the second intermediate structure has an amine group converted to a carboxylic acid group through succinylation of the first intermediate structure.

14. The polymer compound of claim 12, wherein the hydrophobic moiety includes a compound in which polyethylene glycol (PEG) is bound to one end of 1,2-distearoyl-sn-glycero-3-phosphatidylethanolamine (DSPE), and
the linker includes polyethylene glycol (PEG).
